(19) United States
(12) Patent Application Publication
(10) Pub. No.: US 2026/0041543 A1
(43) Pub. Date: Feb. 12, 2026

(54) UNIDIRECTIONALLY-ALIGNED BIOCOMPATIBLE POLYMER FIBER-BASED NERVE CONDUIT AND METHOD OF MANUFACTURING THE SAME

(71) Applicant: KOREA INSTITUTE OF SCIENCE AND TECHNOLOGY, Seoul (KR)

(72) Inventors: Yung Mee JUNG, Seoul (KR); Jin YOO, Seoul (KR); Soo Jung PARK, Seoul (KR)

(73) Assignee: KOREA INSTITUTE OF SCIENCE AND TECHNOLOGY, Seoul (KR)

(21) Appl. No.: 19/247,438

(22) Filed: Jun. 24, 2025

(30) Foreign Application Priority Data
Aug. 7, 2024 (KR) 10-2024-0105404

(51) Int. Cl. A61F 2/02; A61L 27/26; A61L 27/52

(52) U.S. Cl. CPC A61F 2/02; A61L 27/26; A61L 27/52; A61F 2240/001; A61L 2430/32

(57) ABSTRACT

The present invention relates to a nerve conduit including a hollow support including a spring-shaped engraved pattern formed on an inner wall and a biocompatible polymer fiber with a fiber direction aligned parallel to an axonal direction; and an electrically conductive hydrogel applied on the inside or outside of the hollow support, and a method of manufacturing the same.

### CROSS-REFERENCE TO RELATED APPLICATION

This application claims priority to and the benefit of Korean Patent Application No. 2024-0105404, filed on Aug. 7, 2024, the disclosure of which is incorporated herein by reference in its entirety.

### BACKGROUND

### 1. Field of the Invention

The present invention relates to a biocompatible polymer fiber-based nerve conduit unidirectionally aligned through electrospinning using an electric field and a method of manufacturing the same.

### 2. Discussion of Related Art

The critical distance of nerve defect sites is difficult to increase due to the limitations of Schwann cell migration and nerve regeneration because of the slow growth rate and short growth period in nerve regeneration. Accordingly, the development of a nerve conduit that may promote Schwann cell migration and nerve regeneration is necessary. The commercialized nerve conduit is a hollow cylindrical nerve conduit that provides a space in parallel with the direction of the axon, thereby promoting the movement of materials between two disconnected nerve tissues. However, it has limitations as a nerve conduit due to the random arrangement of nerve bundles and the absence of a substance that promotes nerve regeneration. Accordingly, it is necessary to produce a nerve conduit including topographical properties, biochemical properties, and electrical stimulation that can induce the movement of Schwann cells and the promotion of nerve regeneration. In particular, since cell alignment is possible when cells have a pattern structure in the size of nanometers to tens of micrometers based on the cell length and the extracellular matrix size, an aligned structure in the size of nanometers to tens of micrometers is required to control cell alignment, and the alignment direction must be in the same direction as the axonal direction. In addition, actual nerves are tissues that transmit electrical signals, and not only nerves but also the tissue environment has an intrinsic electric field, which guides the tissue formation direction and increases tissue growth, differentiation, and migration to enable tissue recovery, so electrical stimulation may accelerate nerve recovery. Accordingly, an electrically conductive nerve conduit may promote nerve regeneration by improving intrinsic and external electric fields.

### SUMMARY OF THE INVENTION

The present invention is directed to providing a nerve conduit including a hollow support including a spring-shaped engraved pattern formed on an inner wall and a biocompatible polymer fiber with a fiber direction aligned parallel to an axonal direction; and an electrically conductive hydrogel applied on the inside or outside of the hollow support; and the like.

However, the technical tasks to be achieved by the present invention are not limited to the above-mentioned tasks, and other tasks that are not mentioned will be clearly understood by those skilled in the art from the description below.

The present invention provides a nerve conduit including a hollow support including a spring-shaped engraved pattern formed on an inner wall and a biocompatible polymer fiber with a fiber direction aligned parallel to an axonal direction; and an electrically conductive hydrogel applied on the inside or outside of the hollow support.

The biocompatible polymer may include one or more selected from the group consisting of polycaprolactone, polylactide, polyglycolide, polyurethane, polydioxanone, polyethylene glycol, poly(N-isopropylacrylamide-co-acrylic acid), polyvinyl alcohol, polystyrene, and polyester.

The electrically conductive hydrogel may be obtained by gelating one or more selected from the group consisting of gelatin, gelatin methacrylate (GelMA), PEG, polyethylene oxide (PEO), polyhydroxyethyl methacrylate (PHEMA), polyacrylic acid (PAA), PVA, poly(N-isopropylacrylamide) (PNIPAM), polyvinylpyrrolidone (PVP), PLA, PGA, PCL, alginate, carrageenan, chitosan, hydroxyalkyl cellulose, alkyl cellulose, silicone, rubber, agar, carboxyvinyl copolymers, polydioxolane, polyacryl acetate, polyvinyl chloride, collagen, fibrin, Matrigel, and maleic anhydride/vinyl ether.

The electrically conductive hydrogel may further include tannic acid (TA) and polypyrrole.

The nerve conduit may have i) a tensile stress of 2 MPa to 6 MPa at a tensile strain of 200%; ii) a fracture strain of 300% to 500%; iii) an ultimate strength of 4 MPa to 8 MPa; iv) a Young's modulus of 2 MPa to 4 MPa.

The nerve conduit may have an ionic resistance (Zre) of 100 ohms to 1,000 ohms at a frequency of 1 Hz.

The nerve conduit may be for regeneration of central nerves, peripheral nerves, or spinal nerves.

One embodiment of the present invention provides a method of manufacturing a nerve conduit, including: (a) preparing an electrospinning collector including a rod and a conductive wire wound in a spring shape on the rod; (b) manufacturing a hollow support by electrospinning a biocompatible polymer solution onto the electrospinning collector; and (c) applying an electrically conductive hydrogel on the inside or outside of the hollow support.

The method may further include, after Step (b), elongating the manufactured hollow support 1.2 to 5 times by application of tension to manufacture an extended hollow support.

The electrospinning in Step (b) may be performed in a dry state with a humidity of 30% or less, at a spinning speed of 0.1 ml/h to 1.0 ml/h; a spinning distance of 10 cm to 20 cm; an electrospinning collector rotation speed of 10 rpm to 200 rpm; and a voltage of 10 kV to 50 kV.

The method may further include, before Step (c), performing plasma treatment on the hollow support.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a schematic diagram schematically illustrating a method of manufacturing a biocompatible polymer fiber-based nerve conduit unidirectionally aligned through electrospinning using an electric field according to one embodiment of the present invention;

FIG. 2 shows scanning electron microscope (SEM) analysis photographs confirming the directionality of biocompatible polymer fibers in a hollow support extended through tension according to one embodiment of the present invention;

FIG. 3 shows graphs respectively confirming the tensile stress according to the tensile strain, fracture strain, ultimate strength, and Young's modulus in a hollow support extended through tension according to one embodiment of the present invention;

FIG. 4 shows photographs and graphs confirming the alignment of neurons (SH-SY5Y) and Schwann cells (RSC96) according to the directionality of biocompatible polymer fibers in a support extended through tension according to one embodiment of the present invention;

FIG. 5 shows photographs and graphs confirming the degree of differentiation of neural cells (SH-SY5Y) according to the directionality of biocompatible polymer fibers in a support extended through tension according to one embodiment of the present invention;

FIG. 6 shows photographs confirming the degree of electrically conductive hydrogel coating before and after plasma treatment in a hollow support extended through tension according to one embodiment of the present invention through picrosirius red staining;

FIG. 7 shows a graph showing the ion conductivity of an electrically conductive hydrogel applied according to one embodiment of the present invention and measured using a multimeter; and FIG. 8 shows photographs illustrating an analysis of the neural differentiation ability of an electrically conductive hydrogel by culturing neural cells in the electrically conductive hydrogel according to one embodiment of the present invention under electrical stimulation.

### DETAILED DESCRIPTION OF EXEMPLARY EMBODIMENTS

The present inventors confirmed that an electrically conductive nerve conduit with biocompatible polymer fibers further aligned unidirectionally in parallel with an axonal direction may be manufactured by manufacturing a hollow support by electrospinning biocompatible polymer fibers using an optimally designed electrospinning collector, then elongating the support through tension, and coating the inside and outside thereof with an electrically conductive hydrogel, thereby completing the present invention.

Hereinafter, the present invention will be described in detail.

### Unidirectionally-Aligned Biocompatible Polymer Fiber-Based Nerve Conduit

The present invention provides a nerve conduit including a hollow support including a spring-shaped engraved pattern formed on an inner wall and a biocompatible polymer fiber with a fiber direction aligned parallel to an axonal direction; and an electrically conductive hydrogel applied on the inside or outside of the hollow support.

First, the nerve conduit according to the present invention includes a hollow support including a spring-shaped engraved pattern formed on an inner wall and a biocompatible polymer fiber with a fiber direction aligned parallel to an axonal direction.

The hollow support refers to a tube-shaped support, and is characterized in that it has a spring-shaped engraved pattern formed on the inner wall, which may be an engraved pattern corresponding to the electrospinning collector described below.

In addition, the hollow support includes biocompatible polymer fibers, which are aligned unidirectionally and may be aligned in the direction parallel to an axonal direction. This is a result of spinning a biocompatible polymer solution along an electric field generated in the gap/space between a rod and a conductive wire in the electrospinning collector described below. As a result, the migration of Schwann cells and promotion of nerve regeneration may be induced.

Specifically, the biocompatible polymer may include one or more selected from the group consisting of polycaprolactone (PCL), polylactide (PLA), polyglycolide (PGA), polyurethane (PU), polydioxanone (PDO), polyethylene glycol (PEG), poly(N-isopropylacrylamide-co-acrylic acid), polyvinyl alcohol (PVA), polystyrene (PS), and polyester, and is preferably poly(L-lactide-co-ε-caprolactone) (PLCL), but is not limited thereto.

The PLCL is a polymer that may create an environment suitable for regeneration inside a nerve conduit, and when transplanted into mice, it may exhibit a level of regeneration ability similar to that of autologous tissue transplantation in terms of motor function regeneration.

In the PLCL, L-lactide (LA) and ε-caprolactone (CL) may be mixed at a weight ratio of 4:6 to 6:4, preferably at a weight ratio of 4.5:5.5 to 5:5:4.4, and most preferably at a weight ratio of 5:5. In this case, when the mixing ratio of the LA and CL is out of the above-described range, the biodegradation performance may be reduced, and mechanical properties, such as elasticity, tensile strength, and recovery rate, which are unique properties of a PLCL polymer, may be reduced.

The PLCL polymer may have a weight average molecular weight of 100,000 to 123,000 g/mol, preferably 100,000 to 122,000 g/mol, and most preferably 100,000 to 121,000 g/mol. At this time, when the weight average molecular weight of the PLCL polymer is less than 100,000 g/mol, the mechanical rigidity of the biodegradable polymer may be reduced, and conversely, when it exceeds 123,000 g/mol, the biodegradation period may be prolonged, and processing as a tissue engineering support may be difficult due to strong physical properties.

Next, the nerve conduit according to the present invention includes an electrically conductive hydrogel applied on the inside or outside of the hollow support. Specifically, the electrically conductive hydrogel may be obtained by gelating one or more selected from the group consisting of gelatin, gelatin methacrylate (GelMA), PEG, polyethylene oxide (PEO), polyhydroxyethyl methacrylate (PHEMA), polyacrylic acid (PAA), PVA, poly(N-isopropylacrylamide) (PNIPAM), polyvinylpyrrolidone (PVP), PLA, PGA, PCL, alginate, carrageenan, chitosan, hydroxyalkyl cellulose, alkyl cellulose, silicone, rubber, agar, carboxyvinyl copolymers, polydioxolane, polyacryl acetate, polyvinyl chloride, collagen, fibrin, Matrigel, and maleic anhydride/vinyl ether, and considering all physical properties such as viscoelasticity and mechanical strength, it may preferably be GelMA, but is not limited thereto.

The electrically conductive hydrogel may further include tannic acid (TA) and polypyrrole. The TA may form an electron bond with Fe³⁺ of FeCl₃·6H₂O (iron (III) chloride hexahydrate), which is an oxidizing agent used in the synthesis of polypyrrole, and may also form a hydrogen bond with polypyrrole, thereby allowing polypyrrole to penetrate well into the gel. In addition, the polypyrrole serves to provide electrical conductivity to the hydrogel. In this way, by further including TA and polypyrrole in the electrically conductive hydrogel, ion conductivity may be increased, and the neurites of neurons may be better extended.

Through this, the nerve conduit according to the present invention may have: i) a tensile stress of 2 MPa to 6 MPa (preferably 2 MPa to 4 MPa) at a tensile strain of 200%; ii) a fracture strain of 300% to 500% (preferably 350% to 500%); iii) an ultimate strength of 4 MPa to 8 MPa (preferably 6 MPa to 8 MPa); and iv) a Young's modulus of 2 MPa to 4 MPa (preferably 3 MPa to 4 MPa). In other words, the nerve conduit may have optimized physical properties such as elasticity.

In addition, the nerve conduit may have an ionic resistance (Zre) of 100 ohms to 1,000 ohms at a frequency of 1 Hz, and thus is characterized by excellent ionic conductivity.

Accordingly, the nerve conduit may be for regeneration of central nerves, peripheral nerves, or spinal nerves. The nervous system of higher animals may be divided into the central nervous system, peripheral nervous system, and autonomic nervous system, and the central nervous system is a nervous system including the brain and spinal cord. In addition, the peripheral nervous system refers to a nervous system that originates from the central nervous system of the brain or spinal cord and is distributed throughout the body in a branch shape. The nerve conduit may be introduced into a severed or damaged central nerve, peripheral nerve, or spinal nerve site to promote regeneration of the severed or damaged nerve.

### Method of Manufacturing Biocompatible Polymer Fiber-Based Nerve Conduit Unidirectionally Aligned by Electrospinning Using Electric Field

The present invention provides a method of manufacturing a nerve conduit, including: (a) preparing an electrospinning collector including a rod and a conductive wire wound in a spring shape on the rod; (b) manufacturing a hollow support by electrospinning a biocompatible polymer solution onto the electrospinning collector; and (c) applying an electrically conductive hydrogel on the inside or outside of the hollow support.

First, the method of manufacturing a nerve conduit according to the present invention includes preparing an electrospinning collector including a rod and a conductive wire wound in a spring shape on the rod [Step (a)].

An electric field may be generated in the gap/space between the rod and the conductive wire in the electrospinning collector, and a biocompatible polymer solution may be spun along the electric field to unidirectionally align the biocompatible polymer fibers parallel to an axonal direction.

Specifically, the rod may be cylindrical, may have a diameter of 0.5 mm to 10 mm, and may be made of a metallic material with low conductivity such as stainless steel, or may be made of a non-conductive material such as Teflon. In addition, the conductive wire may have a thickness of 0.1 mm to 0.3 mm, and may be made of a metallic material such as copper. At this time, the conductive wire may be wound in a spring shape on the rod, and may be maintained spaced apart at a distance of 0.1 mm to 2 mm.

Next, the method of manufacturing a nerve conduit according to the present invention includes manufacturing a hollow support by electrospinning a biocompatible polymer solution onto the electrospinning collector [Step (b)].

Since the biocompatible polymer has been described above, a redundant description thereof will be omitted. In the biocompatible polymer solution, the concentration of the biocompatible polymer may be 1 (w/w)% to 10 (w/w)%, and as a solvent, hexafluoroisopropanol (HFIP), dimethylformamide (DMF), tetrahydrofuran (THF), dichloromethane (DCM), chloroform, acetone, and the like may be used, and HFIP is preferable, but the present invention is not limited thereto.

The electrospinning is for manufacturing a hollow support including biocompatible polymer fibers unidirectionally aligned parallel to an axonal direction using a biocompatible polymer solution.

Specifically, the electrospinning may be performed in a dry state with a humidity of 30% or less, at a spinning speed of 0.1 ml/h to 1.0 ml/h; a spinning distance of 10 cm to 20 cm; an electrospinning collector rotation speed of 10 rpm to 200 rpm; and a voltage of 10 kV to 50 kV. When the electrospinning is performed under conditions exceeding a humidity of 30%, there is a problem in that biocompatible polymer fibers are manufactured in a bead shape rather than a linear shape.

The hollow support is characterized by having a spring-shaped engraved pattern formed on the inner wall, which may be an engraved pattern corresponding to the electrospinning collector described above. In addition, since a biocompatible polymer solution is spun along an electric field generated in the gap/space between the rod and the conductive wire in the electrospinning collector, the hollow support may include biocompatible polymer fibers unidirectionally aligned parallel to an axonal direction.

After manufacturing a hollow support through the electrospinning, a step of elongating the manufactured hollow support 1.2 to 5 times (preferably 1.2 to 3 times) by application of tension to manufacture an extended hollow support may be further included. By extending the hollow support through such tension, it is possible to optimize physical properties such as elasticity, and there is an advantage of further aligning neurons or Schwann cells according to the directionality of the biocompatible polymer fibers.

Next, the method of manufacturing a nerve conduit according to the present invention includes applying an electrically conductive hydrogel on the inside or outside of the hollow support [Step (c)].

Since the electrically conductive hydrogel has been described above, a redundant description thereof will be omitted.

The method may further include performing plasma treatment on the hollow support, before applying the electrically conductive hydrogel. Due to the plasma treatment, hydroxyl groups may be introduced to the surface of the hollow support to increase hydrophilicity. Accordingly, the plasma-treated hollow support may be pretreated to a state suitable for electrically conductive hydrogel coating which is hydrophilic.

When the electrically conductive hydrogel further includes tannic acid, the TA may be added to the solution before gelation. Specifically, in the solution before gelation, the concentration of the TA may be 0.01 mg/ml to 1 mg/ml. In addition, an initiator such as lithium phenyl-2,4,6-trimethylbenzoylphosphinate (LAP) may be added to the solution before gelation. The TA may form an electron bond with Fe³⁺ of FeCl₃·6H₂O (iron (III) chloride hexahydrate), which is an oxidizing agent used in the synthesis of polypyrrole, and may also form a hydrogen bond with polypyrrole, thereby allowing polypyrrole to penetrate well into the gel Thereafter, the gelation may be performed under ultraviolet conditions for one minute to one hour.

When the electrically conductive hydrogel further includes polypyrrole, as a precursor of the polypyrrole, a pyrrole solution and an oxidizing agent (e.g., FeCl₃·6H₂O (iron (III) chloride hexahydrate)) solution may be added after gelation. Specifically, after the gelation, the hydrogel may be immersed in the pyrrole solution at 30° C. to 40° C. for 1 hour to 20 hours at 10 rpm to 100 rpm, and then an oxidizing agent solution may be added and allowed to react at 1° C. to 10° C. for 1 hour to 20 hours, thereby allowing the polypyrrole to penetrate well into the gel. The polypyrrole serves to impart electrical conductivity to the hydrogel.

As described above, the nerve conduit according to the present invention includes a hollow support including a spring-shaped engraved pattern formed on an inner wall and a biocompatible polymer fiber with a fiber direction aligned parallel to an axonal direction; and an electrically conductive hydrogel applied on the inside or outside of the hollow support, and thus can promote nerve regeneration due to the combination of a unidirectionally-aligned biocompatible polymer fiber and an electrically conductive hydrogel.

In particular, since the nerve conduit according to the present invention is extended through tension, its physical properties such as elasticity are optimized, and neurons or Schwann cells can be further aligned according to the directionality of the biocompatible polymer fibers, so the nerve conduit is particularly effective in promoting nerve regeneration.

Hereinafter, preferred examples are presented to help understand the present invention. However, the examples described below are provided only to help understand the present invention more easily, and the content of the present invention is not limited by the examples described below.

### EXAMPLES

### Example 1

### (1) Electrospinning of Biocompatible Polymer Fibers Using Electrospinning Collector

An electrospinning collector consisting of a cylindrical stainless steel rod with a diameter of 1 mm and a 0.18 mm thick copper conductive wire wound in a spring form on the rod and spaced apart at a distance of 0.3 mm was prepared. Meanwhile, a 1,1,1,3,3,3-hexafluoro-2-propanol (HFIP) solution containing 5 (w/w)% of poly(L-lactide-co-ε-caprolactone) (PLCL) was prepared as a biocompatible polymer solution.

Thereafter, the biocompatible polymer solution was electrospun onto the electrospinning collector. Specifically, 0.8 ml of the biocompatible polymer solution was electrospun onto the electrospinning equipment connected to a dehumidifier to maintain a dry state with a humidity of 30% or less, and at this time, electrospinning was performed at a spinning speed of 0.4 ml/h, a spinning distance of 15 cm, an electrospinning collector rotation speed of 100 rpm, and a voltage of 21 kV.

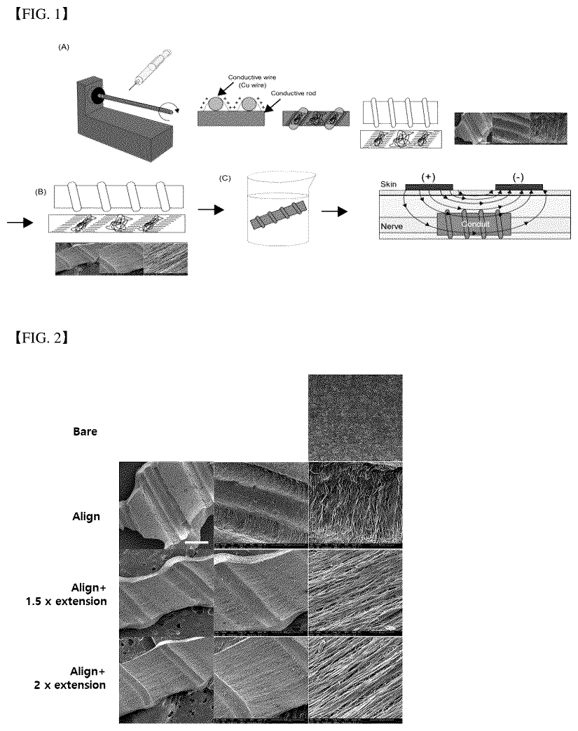

Thereafter, the hollow support on the electrospinning collector was immersed in methanol to separate them from each other. The hollow support separated from the electrospinning collector had the shape of a cylindrical tube with a spring-shaped engraved pattern formed on the inner wall. In addition, biocompatible polymer fibers could be aligned unidirectionally (A of FIG. 1).

### (2) Manufacturing of Hollow Support Extended Through Tension

Tension was applied to both ends of the hollow support manufactured in Example 1 to elongate it 1.5 or 2 times, thereby manufacturing an extended hollow support. Through this, the biocompatible polymer fibers could be made longer, and the fibers could be further aligned unidirectionally (B of FIG. 1 and FIG. 2).

As shown in FIG. 2, the scanning electron microscope (SEM) analysis results showed that in the case of the conventional hollow support (Bare), the biocompatible polymer fiber directionality was not confirmed at all. In particular, it was confirmed that the hollow support that was extended 1.5 or 2 times by applying tension (Align+1.5×extension and Align+2×extension) could further align the biocompatible polymer fibers unidirectionally compared to the hollow support that was not subjected to tension (Align).

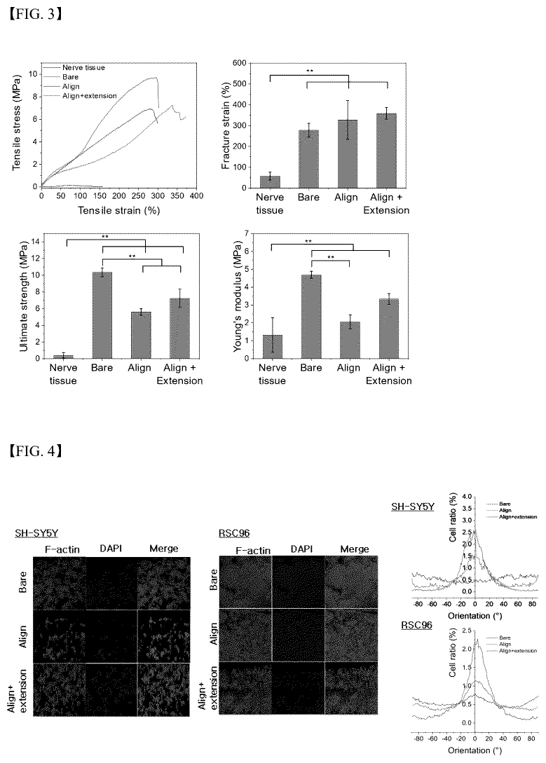

Meanwhile, the tensile stress according to the tensile strain, fracture strain, ultimate strength, and Young's modulus of the hollow support that was extended by elongating it two times by applying tension were measured (FIG. 3).

As shown in FIG. 3, it was confirmed that the hollow support that was extended by elongating it two times by applying tension had a tensile stress of about 3 MPa at a tensile strain of 200%, a fracture strain of about 350%, an ultimate strength of 7 MPa, and a Young's modulus of about 3.3 MPa. Therefore, it can be seen that physical properties such as elasticity were optimized compared to actual nerves (nerve tissue), a conventional hollow support (Bare), or a hollow support that was not subjected to tension (Align).

### Example 2

A hollow support was manufactured in the same manner as in (1) of Example 1, except that an electrospinning collector consisting of a cylindrical stainless steel rod with a diameter of 5 mm and a 0.18 mm thick copper conductive wire wound in a spring form on the rod and spaced apart at a distance of 1 mm was prepared and that 4 ml of a biocompatible polymer solution was electrospun.

Thereafter, the cylindrical tube shape was cut, stretched into a film shape, and tension was applied to both ends to elongate it two times, thereby manufacturing an extended support. Neuronal cells (SH-SY5Y) and Schwann cells (RSC96) were cultured on the support that was elongated two times by applying tension (FIG. 4).

As shown in FIG. 4, it was confirmed that cell alignment varied depending on the directionality of the biocompatible polymer fibers. In particular, it was confirmed that cells were aligned with a significantly higher directionality on the support (Align+extension) that was extended by elongating it two times by applying tension compared to a conventional hollow support (Bare) or a hollow support that was not subjected to tension (Align).

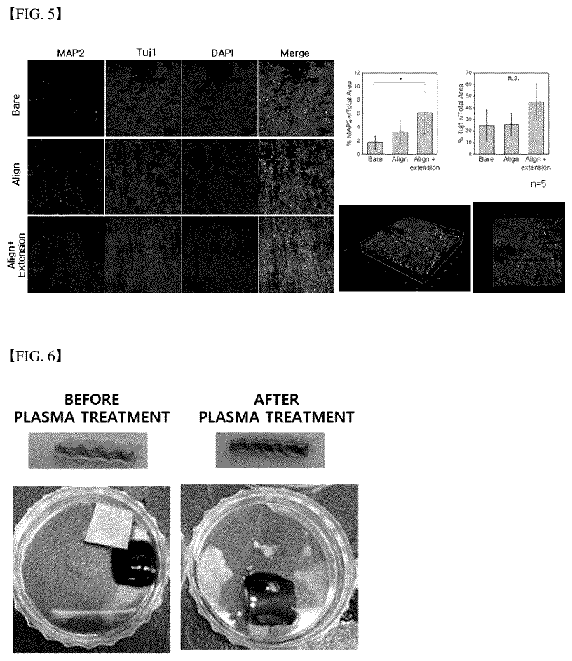

In addition, the degree of differentiation of the neural cells (SH-SY5Y) on the extended support, which was elongated twice by applying tension, was confirmed through neural differentiation marker staining (MAP2, Tuj1, DAPI) (FIG. 5).

As shown in FIG. 5, it was confirmed that neural differentiation occurred depending on the directionality of the biocompatible polymer fibers. In particular, it was confirmed that neural differentiation was significantly improved on the support (Align+extension) that was extended by elongating it two times by applying tension compared to a conventional hollow support (Bare) or a hollow support that was not subjected to tension (Align).

### Example 3

Before performing hydrophilic electrically conductive hydrogel coating on the hollow support that was extended by elongating it two times by applying tension according to Example 1, oxygen plasma treatment was performed under vacuum conditions.

Thereafter, for electrically conductive hydrogel coating, gelatin methacrylate (GelMA) was diluted in water at a concentration of 10 (w/w)%, and 0.1 mg/ml of tannic acid (TA) was added to the solution. In addition, lithium phenyl-2,4,6-trimethylbenzoylphosphinate (LAP) was added as an initiator at a concentration of 0.05 (w/w)%, and a GelMA-TA hydrogel was prepared by gelation under ultraviolet (365 nm) for three minutes.

The GelMA-TA hydrogel was immersed in a 0.01 M pyrrole solution at 37° C. for four hours at 50 rpm, then a 0.25 M FeCl₃·6H₂O (iron (III) chloride hexahydrate) solution was added as an oxidizing agent, and the reaction was performed at 4° C. for 12 hours to infiltrate polypyrrole into the GelMA-TA hydrogel. Thereafter, the hydrogel was washed three times or more in a saline solution to remove byproducts (C of FIG. 1).

Meanwhile, the degree of the electrically conductive hydrogel coating of the hollow support that was extended by elongating it two times by applying tension, before and after the plasma treatment, was shown through picrosirius red staining (FIG. 6).

As shown in FIG. 6, the hollow support that was extended by elongating it two times by applying tension exhibited increased hydrophilicity as hydroxyl groups were introduced to the surface after the plasma treatment, and thus, it was confirmed that a larger amount of the electrically conductive hydrogel was applied.

In addition, the ionic conductivity of the applied electrically conductive hydrogel was measured using a multimeter (FIG. 7).

As shown in FIG. 7, for GelMA-TA, GelMA-PPy, and GelMA-TA-PPy hydrogels containing TA and/or polypyrrole, the ionic resistance (Zre) at a frequency of 1 Hz was significantly lower than that of the simple GelMA hydrogel, indicating that the ionic conductivity was significantly higher.

In addition, the neural differentiation ability of the electrically conductive hydrogel was confirmed by culturing neurons in the applied electrically conductive hydrogel under electrical stimulation. The electrical stimulation apparatus was manufactured using copper plates, polydimethylsiloxane, a cell culture dish, wires, and alligator clips, and the applied electrical stimulation conditions included a free run voltage of 15 V, a frequency of 10 Hz, a delay time of 10 ms, a stimulation width of 10 ms, and a positive electrode, and the electrical stimulation was performed for a total of four times for one hour each day (FIG. 8).

As shown in FIG. 8, it was confirmed that the neurites of the neurons were extended better in the GelMA-PPy or GelMA-TA-PPy hydrogel containing polypyrrole compared to the GelMA-TA hydrogel containing no polypyrrole.

The nerve conduit according to the present invention includes a hollow support including a spring-shaped engraved pattern formed on an inner wall and a biocompatible polymer fiber with a fiber direction aligned parallel to an axonal direction; and an electrically conductive hydrogel applied on the inside or outside of the hollow support, and thus can promote nerve regeneration due to the combination of a unidirectionally-aligned biocompatible polymer fiber and an electrically conductive hydrogel.

In particular, since the nerve conduit according to the present invention is extended through tension, its physical properties such as elasticity are optimized, and neurons or Schwann cells can be further aligned according to the directionality of the biocompatible polymer fibers, so the nerve conduit is particularly effective in promoting nerve regeneration.

The above description of the present invention is for illustrative purposes only, and those skilled in the art will understand that the present invention can be easily modified into other specific forms without changing the technical spirit or essential characteristics of the present invention. Therefore, it should be understood that the above-described embodiments are exemplary in all respects and not restrictive.

What is claimed is:

1. A nerve conduit comprising:
a hollow support including a spring-shaped engraved pattern formed on an inner wall and a biocompatible polymer fiber with a fiber direction aligned parallel to an axonal direction; and
an electrically conductive hydrogel applied on the inside or outside of the hollow support.

2. The nerve conduit of claim 1, wherein the biocompatible polymer includes one or more selected from the group consisting of polycaprolactone, polylactide, polyglycolide, polyurethane, polydioxanone, polyethylene glycol, poly(N-isopropylacrylamide-co-acrylic acid), polyvinyl alcohol, polystyrene, and polyester.

3. The nerve conduit of claim 1, wherein the electrically conductive hydrogel is obtained by gelating one or more selected from the group consisting of gelatin, gelatin methacrylate (GelMA), polyethylene glycol (PEG), polyethylene oxide (PEO), polyhydroxyethyl methacrylate (PHEMA), polyacrylic acid (PAA), polyvinyl alcohol (PVA), poly(N-isopropylacrylamide) (PNIPAM), polyvinylpyrrolidone (PVP), polylactic acid (PLA), polyglycolic acid (PGA), polycaprolactone (PCL), alginate, carrageenan, chitosan, hydroxyalkyl cellulose, alkyl cellulose, silicone, rubber, agar, carboxyvinyl copolymers, polydioxolane, polyacryl acetate, polyvinyl chloride, collagen, fibrin, Matrigel, and maleic anhydride/vinyl ether.

4. The nerve conduit of claim 1, wherein the electrically conductive hydrogel further includes tannic acid and polypyrrole.

5. The nerve conduit of claim 1, wherein the nerve conduit has:
i) a tensile stress of 2 MPa to 6 MPa at a tensile strain of 200%;
ii) a fracture strain of 300% to 500%;
iii) an ultimate strength of 4 MPa to 8 MPa; and
iv) a Young's modulus of 2 MPa to 4 MPa.

6. The nerve conduit of claim 1, wherein the nerve conduit has an ionic resistance (Zre) of 100 ohms to 1,000 ohms at a frequency of 1 Hz.

7. The nerve conduit of claim 1, wherein the nerve conduit is for regeneration of central nerves, peripheral nerves, or spinal nerves.

8. A method of manufacturing a nerve conduit, comprising:
(a) preparing an electrospinning collector including a rod and a conductive wire wound in a spring shape on the rod;
(b) manufacturing a hollow support by electrospinning a biocompatible polymer solution onto the electrospinning collector; and
(c) applying an electrically conductive hydrogel on the inside or outside of the hollow support.

9. The method of claim 8, further comprising, after Step (b), elongating the manufactured hollow support 1.2 to 5 times by application of tension to manufacture an extended hollow support.

10. The method of claim 8, wherein the electrospinning in Step (b) is performed in a dry state with a humidity of 30% or less, at a spinning speed of 0.1 ml/h to 1.0 ml/h; a spinning distance of 10 cm to 20 cm; an electrospinning collector rotation speed of 10 rpm to 200 rpm; and a voltage of 10 kV to 50 kV.

11. The method of claim 8, further comprising, before Step (c), performing plasma treatment on the hollow support.
